(19) United States
(12) Patent Application Publication
(10) Pub. No.: US 2026/0327186 A1
(43) Pub. Date: Oct. 22, 2026

(54) COMPOSITIONS AND METHODS AND USES RELATED THERETO

(71) Applicant: INNOSPEC LIMITED, Ellesmere Port (GB)

(72) Inventors: Tony GOUGH, Chester (GB); Peter Thomas SWALLOW, Liverpool (GB)

(21) Appl. No.: 19/484,543

(22) PCT Filed: May 14, 2024
(86) PCT No.: PCT/GB2024/051255

§ 371 Date: Nov. 14, 2025

(30) Foreign Application Priority Data
May 17, 2023 (GB) 2307390.1

(51) Int. Cl. C07C 303/22; A01N 25/30; C11D 1/28

(52) U.S. Cl. CPC C07C 303/22; A01N 25/30; C11D 1/28

(57) ABSTRACT

A method of preparing an alkyl acyl isethionate surfactant composition, the method including reacting a carboxylic acid of formula R¹COOH or a reactive equivalent thereof with a compound of formula (I):

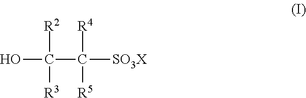

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; and wherein the compound of formula (I) has a percentage renewable carbon index (RCI) of at least 50%.

The present invention relates to alkyl acyl isethionate compounds, compositions comprising such compounds and methods and uses relating thereto.

Alkyl acyl isethionate compounds are common, versatile surfactants that can be used in a variety of applications. They are particularly useful in personal care applications due to their low irritancy potential, desirable foaming properties, high solubility and stability.

However current commercial compositions comprising such surfactants are derived from petrochemical sources.

Environmental concerns are becoming increasingly important to consumers with many seeking to purchase household and cosmetic products having a reduced environmental impact.

Therefore it is highly desirable to provide mild surfactants that can be prepared from renewable carbon sources.

In this specification when we refer to renewable carbon sources we mean to include all carbon sources that are not directly obtained from petrochemicals. However the term is intended to encompass recovered or recycled carbon that may originally have been obtained from such sources.

Thus renewable carbon sources may include those classed as green (plant derived), blue (marine derived), purple (deriving from carbon capture) or grey (from plastics or pyrolysis of other material).

According to the first aspect of the present invention there is provided a method of preparing an alkyl acyl isethionate surfactant composition, the method comprising reacting a carboxylic acid of formula R¹COOH or a reactive equivalent thereof with a compound of formula (I):

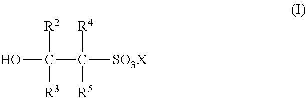

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; and wherein the compound of formula (I) has a percentage renewable carbon index (RCI) of at least 50%.

The method of the first aspect of the present invention relates to a method of preparing an alkyl acyl isethionate composition. The alkyl acyl isethionate composition provided may consist essentially of an alkyl acyl isethionate compound or it may comprise one or more further components. The one or more further components may, for example, include by-products, residual starting materials, impurities or solvents.

According to a second aspect of the present invention there is provided a surfactant composition comprising one or more alkyl acyl isethionate compounds of formula (II):

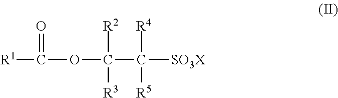

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source.

In some preferred embodiments the surfactant composition of the second aspect may be provided by the method of the first aspect.

According to a third aspect of the present invention there is provided a compound of formula (II):

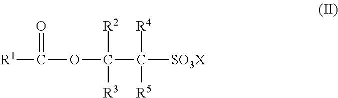

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source.

According to a fourth aspect of the present invention there is provided a compound of formula (I):

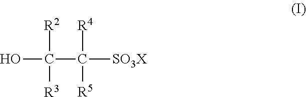

wherein X is hydrogen, a metal ion or an optionally substituted ammonium ion; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms in the compound are from a renewable source Preferred features of the first, second, third and fourth aspects will now be described.

References herein to the surfactant composition of the present invention unless otherwise stated may refer to the surfactant composition of the second aspect and/or the surfactant composition provided by the first aspect.

R¹ represents an optionally substituted C₄-C₃₆ hydrocarbyl group, R², R³, R⁴ and R⁵ each independently represents hydrogen or a substituted or unsubstituted C₁-C₄ alkyl group, provided that at least one of R², R³, R⁴ and R⁵ is not hydrogen, and X represents a cation.

Suitably R¹ represents an optionally substituted C₄-C₃₆ alkyl, C₄-C₃₆ alkenyl, C₆-C₁₂ aryl or C₈-C₂₂ alkyl-C₆-C₁₂ aryl group. More suitably, R¹ represents an optionally substituted C₄-C₃₆ alkyl or C₄-C₃₆ alkenyl group, especially an optionally substituted C₄-C₃₆ alkyl group. Most suitably, R¹ represents a C₄-C₃₆ alkyl or C₄-C₃₆ alkenyl group, especially a C₄-C₃₆ alkyl group.

Suitably R¹ represents an optionally substituted C₄-C₃₆ alkyl or C₄-C₃₆ alkenyl group, such as an optionally substituted C₈-C₁₈ alkyl or C₈-C₁₈ alkenyl group.

Suitably R¹ represents a C₄-C₃₆ alkyl or C₄-C₃₆ alkenyl group, such as a C₈-C₁₈ alkyl or C₈-C₁₈ alkenyl group.

Suitably R¹ represents an optionally substituted C₅-C₃₀ alkyl group, such as an optionally substituted C₇-C₂₄ alkyl group, for example an optionally substituted C₇-C₂₁ alkyl group, preferably an optionally substituted C₇-C₁₇ alkyl group.

Suitably R¹ represents a C₅-C₃₀ alkyl group, such as a C₇-C₂₄ alkyl group, for example a C₇-C₂₁ alkyl group, preferably a C₇-C₁₇ alkyl group.

R¹ is suitably provided by a fatty acid (i.e. an acid of formula R¹COOH). Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, C₈ caprylic acid, C₁₀ capric acid and C₁₈ stearic and oleic acid.

R¹ may be provided by one or more naturally occurring fatty acids and/or of one or more synthetic fatty acids. For example, R¹ may consist essentially of the residue of a single fatty acid.

Most preferably R¹ is provided by a naturally occurring and/or renewable fatty acid.

Examples of carboxylic acids from which R¹ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. R¹ may also be derived from fatty acids obtained via fermentation or general biotechnological processes, or from waste vegetable oils. These may be obtained by chemical or enzymatic routes.

Suitably R¹ is provided by coco acid, mixed fatty acids derived from coconut oil or the mixed fatty acids derived from palm kernel oil. Suitable coconut derived feedstocks include coconut fatty acid, coconut oil, coconut oil methyl esters, virgin coconut oil, refined bleached and deodorised coconut oil, ‘distilled and topped’ hardened coconut fatty acid and methyl esters thereof. Also useful are palm oil, hydrogenated palm kernel oil derived C₁₂-C₁₈ fatty acids (hardened and topped fatty acid) and methyl esters thereof. In some embodiments, R¹ predominantly comprises the residue of a saturated fatty acid having 12 carbon atoms.

In some embodiments only a single acyl alkyl isethionate compound of formula (II) may be present in the surfactant composition of the present invention. In some embodiments a mixture of two or more acyl alkyl isethionate surfactants of formula (II) may be present.

Each of R², R³, R⁴ and R⁵ independently represents hydrogen or a C₁-C₄ alkyl group and wherein at least one of R², R³, R⁴ and R⁵ is not hydrogen.

When any of R², R³, R⁴ and R⁵ represents an optionally substituted C₁-C₄ alkyl group, the alkyl group is suitably n-propyl, ethyl or methyl, such as ethyl or methyl, most preferably methyl.

Preferably one of the groups R², R³, R⁴ and R⁵ represents an optionally substituted C₁-C₄ alkyl group and the remaining groups represent hydrogen. For example, R² may represent an optionally substituted C₁-C₄ alkyl group and R³, R⁴ and R⁵ may all represent hydrogen. For example, R⁴ may represent an optionally substituted C₁-C₄ alkyl group and R², R³ and R⁵ may all represent hydrogen.

Preferably, R² represents a C₁-C₄ alkyl group and R³, R⁴ and R⁵ all represent hydrogen. Preferably, R⁴ represents a C₁-C₄ alkyl group and R², R³ and R⁵ all represent hydrogen.

Most preferably, R² represents a methyl group and R³, R⁴ and R⁵ all represent hydrogen. Most preferably, R⁴ represents a methyl group and R², R³ and R⁵ all represent hydrogen.

Suitably X represents hydrogen, a metal cation or an optionally substituted ammonium cation, preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable metal cations include alkali metal cations, for example sodium, lithium and potassium cations, and alkaline earth metal cations, for example calcium and magnesium cations. Suitably, X represents hydrogen, an alkali metal cation or an optionally substituted ammonium cation. Preferably, X represents a zinc, potassium or sodium cation. Most preferably, X represents a sodium cation.

The skilled person will appreciate that when X is a divalent metal cation two moles of anion will be present for each mole of cation.

In some preferred embodiments the surfactant composition of the present invention may comprise the reaction product of sodium methyl isethionate and a fatty acid, that is a compound of formula R¹COOCHR²CHR⁴SO₃X in which one of R² and R⁴ is methyl and the other is hydrogen. Mixtures of these isomers may be present.

The surfactant composition of the present invention may include a mixture of more than one acyl alkyl isethionate compound of formula (II). For example, an isomeric mixture of acyl alkyl isethionate surfactants of formula (II) may be present. Such a mixture may include, for example an acyl alkyl isethionate surfactant in which R² represents a C₁-C₄ alkyl group (suitably methyl) and R³, R⁴ and R⁵ are all hydrogen and an acyl alkyl isethionate surfactant in which R⁴ represents a C₁-C₄ alkyl group (suitably methyl) and R², R³ and R⁵ are all hydrogen.

In particular, the surfactant composition of the present invention may comprise a mixture of isomers, that is a compound of formula R¹COOCH₂CHR⁴SO₃X in which R⁴ represents a C₁-C₄ alkyl group (preferably methyl) and a compound of formula R¹COOCHR²CH₂SO₃X in which R² represents a C₁-C₄ alkyl group (preferably methyl).

Suitably such mixtures comprise approximately 90% of compounds in which R² is methyl and R⁴ is hydrogen and approximately 10% of compounds in which R² is hydrogen and R⁴ is methyl.

Preferably the surfactant composition provided by the present invention comprises one or more acyl alkyl isethionate compounds of formula (II) selected from sodium lauroyl methyl isethionate, sodium cocoyl methyl isethionate, sodium oleoyl methyl isethionate and sodium myristoyl methyl isethionate. Sodium lauroyl methyl isethionate (SLMI) is especially preferred.

In the first aspect of the present invention an acid of formula R¹COOH or a reactive equivalent thereof is reacted with a compound of formula (I). The reaction may be carried out by any suitable means. Such means will be well known to the person skilled in the art. Suitable methods include those described in WO94/09763, WO2005/075623 and CN115193475.

In the method of the first aspect a carboxylic acid of formula R¹COOH or a reactive equivalent thereof is reacted with a compound of formula (I).

By a reactive equivalent thereof we mean to refer to a compound which reacts in the same way as a carboxylic acid, for example, an acid chloride or anhydride.

In some embodiments the method of the first aspect involves reaction of an acid chloride of formula R¹COOCl with a compound of formula (I). Suitable reactions of this type are described, for example in WO94/09763.

In some embodiments the method of the first aspect involves a direct esterification reaction between the acid of formula R¹COOH and the ester of formula (I). In some such embodiments the reaction may be carried out in the presence of a catalyst. Suitable catalysts will be known to the person skilled in the art and include acids and bases.

Preferred catalysts for use in the method of the first aspect include alkylisethionic acids; salts of hydroxyalkane sulfonates; methane sulfonic acid; p-toluene sulfonic acid; inorganic acids, for example sulfuric acid, phosphoric acid, phosphorous acid, hypophosphoric acid, boric acid or their anhydrides; heavy metal salts, for example zinc sulfate, zirconium sulfate, zinc isethionate, zinc alkylisethionates, zinc cocoate, zinc citrate, zinc borate, aluminum sulfate, titanium sulfate or tungsten phosphate; metal oxides, for example zinc oxide, aluminum oxide, magnesium oxide, cerium oxide, zirconium oxide or lanthanum oxide; and organic acids, for example citric acid and glycolic acid. Metal based catalysts are preferred.

An especially preferred catalyst is the compound of formula (I) in which X is hydrogen.

In one embodiment, a suitable catalyst may be prepared by reacting molten fatty acid with a metal oxide or hydroxide (such as zinc or iron oxide or hydroxide), acid solution and hydrogen peroxide. Such a catalyst is described in CN115193475.

Suitable conditions for carrying out the method of the first aspect will be known to the person skilled in the art.

In some preferred embodiments the acid of formula R¹COOH or a reactive equivalent thereof is directly reacted with a compound of formula (I) without a solvent.

In some embodiments the reaction is carried out in the presence of a solvent. Suitable solvents will be known to the person skilled in the art and include water, alkylene glycols (e.g. ethylene glycol and propylene glycol) and aprotic solvents for example alpha omega bis alkylated polyalkylene glycols (e.g. glymes).

A preferred solvent is water.

The method of the first aspect involves reaction of a compound of formula (I) with an acid of formula R¹COOH or a reactive equivalent thereof.

The compound of formula (I) may be obtained by reacting one or more alkylene oxides with bisulfite or sulfite, preferably an aqueous solution of bisulfite. In some embodiments the bisulfite may be prepared in situ by reaction of a hydroxide (e.g. sodium hydroxide) with sulfur dioxide. Suitable methods to prepare the compounds of formula (I) are described in WO2005/075623.

The compound of formula (I) has a percentage renewable carbon index (% RCI) of at least 50%.

Preferably the compound of formula (I) has a percentage renewable carbon index (% RCI) of at least 60%, preferably at least 70%, more preferably at least 80%, preferably at least 90%, suitably at least 98%.

Preferably the compound of formula (I) has a percentage renewable carbon index (% RCI) of from 95 to 100%, preferably from 99 to 100%.

The renewable carbon index (RCI) for a component is calculated according to ISO 16128-1:2016, wherein the number of carbon atoms derived from renewable sources is divided by the total number of carbon atoms in a component. The total RCI of the composition is a weighted average of the RCI of each carbon-containing surfactant in the composition. The % RCI is obtained by multiplying the RCI by 100.

This means that in preferred embodiments substantially all of the carbon atoms in the compound of formula (I) are from renewable sources.

Preferably in the method of the first aspect the carboxylic acid of formula R¹COOH or reactive equivalent thereof has a percentage renewable carbon index (% RCI) of at least 50%.

Preferably the carboxylic acid of formula R¹COOH or reactive equivalent thereof has a percentage renewable carbon index (% RCI) of at least 60%, preferably at least 70%, more preferably at least 80%, preferably at least 90%, suitably at least 98%.

Preferably the carboxylic acid of formula R¹COOH or reactive equivalent thereof has a percentage renewable carbon index (% RCI) of from 95 to 100%, preferably from 99 to 100%.

In the compound of formula (II) at least 50% of the carbon atoms provided by the moiety CR²R³CR⁴R⁵SO₃X in the compound of formula (II) are from a renewable source.

Preferably at least 60% of the carbon atoms provided by the moiety CR²R³CR⁴R⁵SO₃X in the compound of formula (II) are from a renewable source, preferably at least 70%, more preferably at least 80%, preferably at least 90%, suitably at least 98%.

Preferably from 95 to 100% or 98 to 100% of the carbon atoms provided by the moiety CR²R³CR⁴R⁵SO₃X in the compound of formula (II) are from a renewable source.

In preferred embodiments at least 50% of the carbon atoms provided by the moiety R¹ in the compound of formula (II) are from a renewable source.

Preferably at least 60% of the carbon atoms provided by the moiety R¹ in the compound of formula (II) are from a renewable source, preferably at least 70%, more preferably at least 80%, preferably at least 90%, suitably at least 98%.

Preferably from 95 to 100% or 98 to 100% of the carbon atoms provided by the moiety R¹ in the compound of formula (II) are from a renewable source.

Preferably the or each compound of formula (II) provided by the present invention has a percentage renewable carbon index (% RCI) of at least 50%.

Preferably the or each compound of formula (II) has a percentage renewable carbon index (% RCI) of at least 60%, preferably at least 70%, more preferably at least 80%, preferably at least 90%, suitably at least 98%.

Preferably the or each compound of formula (II) has a percentage renewable carbon index (% RCI) of from 95 to 100%, preferably from 99 to 100%.

One way in which the percentage renewable carbon index of a component can be measured is described in example 1.

Another suitable method that can be used to measure the RCI for green, blue, or other biogenic sources of carbon is that described in ASTM D6866.

The surfactant compositions of the present invention may be provided by any suitable renewable source of carbon.

Suitable renewable sources of carbon include those known as grey, purple, green or blue sources of carbon. As will be understood by the skilled person purple carbon is developed from carbon dioxide drawn from industrial emissions; green carbon is obtained from plants and other biological sources; blue carbon is derived from marine sources, for example algae; and grey carbon is recovered from waste materials.

Examples of suitable sources of grey carbon which may be useful herein include products from the pyrolysis of waste, for example plastic waste, biomass for example agricultural waste, forestry waste, waste cooking oils, algae waste, used tyres or waste rubber. Examples of waste plastic which may be pyrolysed to provide carbon for use herein include low density polyethylene, high density polyethylene, ultra-high density polyethylene, polypropylene, polystyrene, polyethylene terephthalates (PET), rubber (e.g. from tyres), polyacrylate and polynitrile.

Examples of suitable sources of purple carbon which may be useful herein include materials developed from carbon captured from air (Direct Air Capture) or captured from the flue stacks during the combustion of fossil fuels (Point Source Capture). Point Source Capture may be carried out for example at power stations or steel manufacturing mills. An example is a process developed by Lanzatech which captures flue gases from heavy industry flue stacks and ferments the gases using certain microbes into feedstocks such as ethanol which can be further converted into feedstocks which can be used in the present invention.

Examples of suitable sources of green carbon which may be useful herein include plants which produce oils, sugar, composts and digestate materials such as lignocellulose. Various methods can be used to convert these green carbon derivatives into feedstocks that can be used in the present invention. For example, ethanol obtained from fermentation of sugars can be converted into propylene which can be converted into propylene oxide. A further example is the conversion of lignocellulose (a waste biomass from trees, bushes and grasses) by thermal catalytic processes into bionaptha. This can be processed further (cracked) into alkene feedstocks such as propylene which can be converted into propylene oxide. Propylene oxide is used to make sodium methyl isethionate.

Examples of suitable sources of blue carbon which may be useful herein include algae and carbon stored in the vegetation and soils of mangroves, salt marshes, and seagrasses. For example, cracking of lipids derived from algae can give alkenes, for example propylene which can be converted into propylene oxide and used to make sodium methyl isethionate.

The surfactant composition of the present invention may be provided by a mixture of carbon sources.

The surfactant composition of the present invention may be provided in solid or liquid form.

In some embodiments the surfactant composition is provided as a powder.

In some embodiments the surfactant composition is provided as an aqueous solution. Such an aqueous solution may be clear or opaque.

In embodiments in which the reaction to form the surfactant composition is carried out under aqueous conditions the mixture obtained may be used directly without purification. In other such embodiments the resultant mixture may be spray dried.

As mentioned above the surfactant composition provided by the present invention may comprise one or more further components such as impurities, byproducts or residual staring materials.

Preferably the surfactant composition provided by the present invention (including any impurities, byproducts or residual staring materials) has a percentage renewable carbon index (% RCI) of at least 50%. Preferably the surfactant composition has a percentage renewable carbon index (% RCI) of at least 60%, preferably at least 70%, more preferably at least 80%, preferably at least 90%, suitably at least 98%.

In preferred embodiments the surfactant composition provided by the present invention comprises less than 30 wt % free fatty acids based on the total mass of solids present in the composition, preferably less than 20 wt %, more preferably less than 15 wt %, suitably less than 10 wt %.

In preferred embodiments the surfactant composition provided by the present invention comprises less than 30 wt % sodium chloride based on the total mass of solids present in the composition, preferably less than 20 wt %, more preferably less than 15 wt %, suitably less than 10 wt %, for example less than 5 wt %.

In some preferred embodiments the surfactant composition provided by the present invention comprises less than 1 wt % sodium chloride, preferably less than 0.5 wt %, more preferably less than 0.1 wt %, suitably less than 0.01 wt %, for example less than 0.001 wt %.

The surfactant composition provided by the present invention may find utility in a wide variety of applications.

According to a fifth aspect of the present invention there is provided the use of a surfactant composition comprising one or more alkyl acyl isethionate compounds of formula (II):

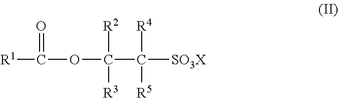

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source; in toilet care, automatic dishwashing, manual dishwashing, laundry, fabric care, kitchen care, carpet cleaning, air fresheners, vehicle care, polishing products, machine cleaning and maintenance, pesticides, insecticides, fungicides, herbicides, oilfield chemical applications, marine applications, personal care or institutional/industrial cleaning.

According to a sixth aspect of the present invention there is provided a formulated composition comprising:
a surfactant composition comprising one or more alkyl acyl isethionate compounds of formula (II):

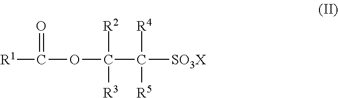

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source; and
one or more further components.

Preferred features of the fifth and sixth aspects of the present invention are as defined in relation to the first, second, third and fourth aspects.

The formulated composition of the sixth aspect comprises a surfactant composition and one or more further components.

By “formulated composition” we mean to refer to a chemical composition which contains multiple components which have been mixed together. The formulated composition is suitably prepared to contain specific ingredients and has a particular purpose. For example the formulated composition may be a cleaning composition or a personal care composition.

The types of components that are typically present in such formulated composition will be known to the person skilled in the art.

For example laundry and dishwashing compositions typically comprise ingredients such as surfactants, builders, bleaches, bleach activators, redeposition additives, dye transfer inhibitors, enzymes, colorants and fragrances.

Personal care compositions typically comprise ingredients such as surfactants (including anionic, amphoteric, nonionic and cationic surfactants); conditioning agents (including quaternary ammonium compounds, cationic polymers, cationic conditioning polymers, silicones, synthetic or natural oils or resins etc), fatty alcohols, electrolytes or other rheology modifiers, opacifying/pearlising agents, scalp benefit agents, fragrances, dyes, UV filters, penetration enhancers (eg, propylene carbonate, benzyl alcohol etc), preservatives, antioxidants, emulsifiers, pH adjusting agents and buffers and styling polymers (eg, polyvinylpyrrolidone etc).

Preferably the one or more further components are selected from chelating agents, preservatives, pH modifiers, hydrotropes and further surfactants.

The formulated composition may comprise a chelating agent. Suitable chelating agents include ethylenediamine-N,N′-disuccinic acid, methylglycinediacetic acid, glutamic acid N,N-diacetic acid, imino disuccinic acid, diethylene triamine pentaacetic acid, ethylenediamine tetraacetic acid, diethylenetriamine penta methylene phosphonic acid, etidronic acid and anions, salts and mixtures thereof.

Preferred chelating agents are biodegradable chelating agents for example ethylenediamine-N,N′-disuccinic acid, methylglycinediacetic acid, glutamic acid N,N-diacetic acid, imino disuccinic acid and anions and mixtures thereof. Ethylenediamine-N,N′-disuccinic acid (EDDS) is especially preferred. The skilled person will appreciate that polycarboxylic acid chelating agents may be present as the free acid or a salt thereof.

The formulated composition may comprise a preservative. Suitable preservatives will be known to the person skilled in the art and include sodium benzoate, potassium sorbate, sorbic acid, phenoxyethanol, benzyl alcohol, DMDM hydantoin, imidazolidinyl urea, methylchloroisothiazolinone, methylisothiazolinone, salicylic acid, benzyl salicylate, methylparaben, propylparaben and caprylyl glycol. A preferred preservative for use herein is sodium benzoate.

The formulated composition may comprise a hydrotrope. Suitable hydrotropes will be known to the person skilled in the art and include propylene glycol, hexylene glycol, glycerine, sorbitol, xylene sulfonates, cumene sulfonates, ethanol, urea, dipropylene glycol. A preferred hydrotrope for use herein is sorbitol.

The formulated composition may comprise a pH modifier. Suitable pH modifiers will be known to the person skilled in the art and include lactic acid, potassium hydroxide sodium hydroxide, sodium carbonate, triethanolamine and sodium gluconate. A preferred pH modifier is citric acid.

Preferably the formulated composition of the present invention has a pH of from 3 to 9, preferably 4 to 8, for example 4.5 to 7.5.

In some embodiments the composition has a pH of 4.5 to 5.5.

In some embodiments the composition has a pH of 6.5 to 7.5.

The formulated composition of the invention may comprise one or more further surfactants.

Such surfactants may be selected from anionic surfactants, cationic surfactants, non-ionic surfactants, amphoteric or zwitterionic surfactants, and mixtures thereof. The selection of suitable further surfactants for use in the composition of the present invention is within the competence of the person skilled in the art.

Suitable anionic surfactants for use in formulated composition of the present invention include salts of C₁₂-C₁₈ carboxylic acids, ethoxylated carboxylic acids, ester carboxylates and ethoxylated ester carboxylates and sarcosinates. Other suitable anionic surfactants include sulfates and sulfonates, for example alkyl sulfates, alkyl ether sulfates, alcohol sulfates, alcohol ether sulfates, α-olefin sulfonates, linear alkyl sulfonates; and phosphate esters.

Suitable anionic surfactants may be selected from salts of fatty acids; alkali metal salts of mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, non-acylated alkyl isethionates; fatty acid taurates; acyl taurates; amino acid surfactants such as glutamates and glycinates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids and salts of lipoamino acids. Particularly exemplary salts of the above, where applicable, are the sodium, potassium, ammonium, magnesium and triethanolamine salts. Suitable ammonium cations include those derived from alkyl amines and alkanolamines.

Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Preferred anionic surfactants are selected from salts of fatty acids; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, non-acylated alkyl isethionates; fatty acid taurates; acyl taurates; amino acid surfactants such as glutamates and glycinates; products of condensation of fatty acids with oxy- and aminoalkanesulfonic acids; alkyl and acyl sarcosinates; sulfoacetates; alkyl phosphates; alkyl phosphate esters; acyl lactates; and salts of lipoamino acids. Particularly exemplary salts of the above, where applicable, are the sodium, potassium, ammonium, magnesium and triethanolamine salts. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable sulfoacetates include acyl sulfoacetates, particularly sodium acyl sulfoacetates.

Suitable glutamate surfactants include acyl glutamates.

Preferred additional anionic detersive surfactants for use in the formulated compositions of the present invention include alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Suitable non-ionic surfactants for use in the formulated compositions of the present invention include alcohol alkoxylates such as alcohol ethoxylates, alcohol propoxylates, and ethylene oxide/propylene oxide copolymer derived surfactants, aliphatic esters, aromatic esters, sugar esters, especially sorbitan esters, alkyl polyglucosides, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides and amineoxides.

Suitable non-ionic surfactants may be selected from the following: reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl (C₆-C₂₂) phenol-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a polysaccharide amide; lactobionamides; and alkyl polysaccharide nonionic surfactants, for example alkylpolyglycosides.

Suitable cationic surfactants for use in the formulated compositions of the present invention are typically based on fatty amine derivates or phosphonium quaternary ions, and quaternary ammonium compounds. Polymeric cationic surfactants may also be used.

Suitable cationic surfactants for use in the formulated compositions of the present invention include tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate.

Examples of suitable cationic surfactants include quaternary ammonium compounds, particularly trimethyl quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen (e.g. bromide), acetate, citrate, lactate, glycolate, phosphate, nitrate, sulfate, or alkylsulfate.

Further suitable cationic surfactants include those materials having the CTFA designations Quaternium-5, Quaternium-31 and Quaternium-18. Mixtures of any of the foregoing materials may also be suitable. A particularly useful cationic surfactant for use as a hair conditioning agent is cetyltrimethylammonium chloride, available commercially, for example as GENAMIN CTAC, ex Hoechst Celanese.

Salts of primary, secondary, and tertiary fatty amines are also suitable cationic surfactants.

The alkyl groups of such amines preferably have from 12 to 22 carbon atoms, and can be optionally substituted.

Useful cationic surfactants include amido substituted tertiary fatty amines, in particular tertiary amines having one C₁₂ to C₂₂ alkyl or alkenyl chain. Such amines include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachid amidopropyldiethylamine, arachidamidoethyldiethylamine, arachidamidoethyldimethylamine, diethylaminoethylstearamide.

Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

These amines are typically used in combination with an acid to provide the cationic species. Suitable acids include L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid.

Other useful cationic amine surfactants include those disclosed in U.S. Pat. No. 4,275,055.

Suitable polymeric cationic surfactants include polyquaternium-7, polyquaternium-10, polyquaternium-11, guar hydroxypropyltrimonium chloride, and hydroxypropyl guar hydroxypropyltrimonium chloride.

Suitable amphoteric surfactants for use in the formulated compositions of the present invention include those based on fatty nitrogen derivates and those based on betaines.

Suitable amphoteric or zwitterionic surfactants may be selected from betaines, for example alkyl betaines, alkylamidopropyl betaines, for example cocamidopropyl betaine, alkylamidopropyl hydroxy sultaines, alkylamphoacetates, alkylamphodiacetates, alkyl propionates, alkylamphodipropionates, alkylamphopropionates, alkyliminodipropionates and alkyliminodiacetate.

Amphoteric or zwitterionic surfactants for use in compositions of the fifth aspect may include those which have an alkyl or alkenyl group of 7 to 22 carbon atoms and comply with an overall structural formula:

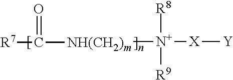

where R⁷ is alkyl or alkenyl of 7 to 22 carbon atoms; R⁸ and R⁹ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂ or —SO₃.

Amphoteric or zwitterionic surfactants may include simple betaines of formula:

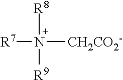

and amido betaines of formula:

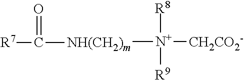

where m is 2 or 3.

In both formulae R¹, R⁸ and R⁹ are as defined previously. R⁷ may, in particular, be a mixture of C₁₂ and C₁₄ alkyl groups derived from coconut so that at least half, preferably at least three quarters, of the groups R⁷ has 10 to 14 carbon atoms. R⁸ and R⁹ are preferably methyl.

Amphoteric or zwitterionic surfactants may include sulfobetaines of formula:

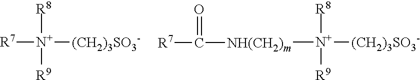

where m is 2 or 3, or variants of these in which
—(CH₂)₃SO₃⁻ is replaced by

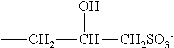

where R¹, R⁸ and R⁹ in these formulae are as defined previously.

Amphoteric or zwitterionic surfactants may include amphoacetates and diamphoacetates. Amphoacetates generally conform to the following formula:

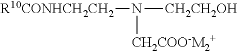

Diamphoacetates generally conform to the following formula:

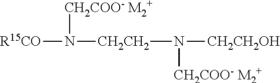

where R¹⁰ is an aliphatic group of 8 to 22 carbon atoms and M₂⁺ is a cation such as sodium, potassium, ammonium, or substituted ammonium.

Suitable acetate-based surfactants include lauroamphoacetate; alkyl amphoacetate; sodium alkyl amphoacetate; cocoampho(di)acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate.

Suitable betaine surfactants include alkylamido betaine; alkyl betaine, C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, Ca amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow aminobetaine.

Suitable glycinate surfactants include acyl glycinates such as cocoamphocarboxyglycinate; tallowamphocarboxygycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C_{10/12} fatty acid amidoethyl-N-(2-hydroxyethyl)-glycinate; N—C_{12/18}-fatty acid amidoethyl-N-(2-hydroxyethyl)-glycinate; dihydroxyethyl tallow glycinate.

Preferred acetate-based amphoteric surfactants include sodium lauroamphoacetate, disodium lauroamphoacetate and mixtures thereof.

Preferred betaine surfactants include cocoamidopropyl betaine.

Preferred sultaine surfactants include cocoamidopropylhydroxy sultaine.

The formulated composition comprises a surfactant composition and optionally one or more further surfactants.

Preferred further surfactants for inclusion in the formulated compositions of the present invention include those which are derived from renewable sources.

The total RCI of the formulated composition is a weighted average of the RCI of each carbon-containing surfactant in the formulated composition. The % RCI is obtained by multiplying the RCI by 100. The term “surfactant” is well understood by a person skilled in the art and such a skilled person would readily understand which components of the formulated composition of the sixth aspect are surfactants.

Preferably total percentage RCI of the formulated composition is at least 30%, preferably at least 40%, more preferably at least 50%, suitably at least 60%, preferably at least 70% or at least 80%.

In some embodiments the total percentage RCI of the formulated composition is more than 85%, for example 90 to 100%, 95 to 100% or 98 to 100%.

The formulated composition of the present invention may be prepared by admixing the surfactant composition with the one or more further components. The preparation of formulated compositions can be simplified by the addition of a multi-component concentrate that contains more than one surfactant. Such materials can reduce the number of steps involved in the preparation of a formulated composition.

According to a seventh aspect of the present invention there is provided a precursor concentrate composition comprising:
a surfactant composition comprising one or more alkyl acyl isethionate compounds of formula (II):

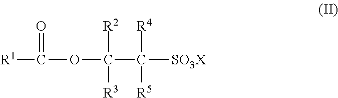

wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source; and
one or more further surfactants.

Preferred features of the seventh aspect of the present invention are as defined in relation to the first, second, third, fourth fifth and sixth aspects.

In particular preferred further surfactants for use in the precursor concentrate composition are as defined in relation to the sixth aspect.

Preferably the precursor concentrate composition comprises a total amount of from 20 to 60 wt % active surfactants, preferably from 30 to 50 wt %.

The precursor concentrate composition may be provided in any suitable form. In particularly preferred embodiments the precursor concentrate composition is a flowable liquid.

Preferred further surfactants for inclusion in the precursor concentrate compositions of the present invention include those which are derived from renewable sources.

The total RCI of the precursor concentrate composition is a weighted average of the RCI of each carbon-containing surfactant in the precursor concentrate composition.

Preferably total percentage RCI of the precursor concentrate composition is at least 30%, preferably at least 40%, more preferably at least 50%, suitably at least 60%, preferably at least 70% or at least 80%.

In some embodiments the total percentage RCI of the precursor concentrate composition is more than 85%, for example 90 to 100%, 95 to 100% or 98 to 100%.

In preferred embodiments of the present invention the formulated composition of the sixth aspect of the present invention is a personal care composition.

The formulated personal care composition may comprise one or more foaming agents, emollients, dyes, fragrances, chelating agents, thickeners, conditioners, thickening agents, preservatives, antimicrobials, sequestrants, vitamins and derivatives thereof, sunscreens, desquamation actives, anti-wrinkle/anti-atrophy actives, anti-oxidants, skin soothing agents/skin healing agents, skin lightening agents, skin tanning agents, anti-acne medicaments, essential oils, sensates, pigments, pearlescent agents and interference pigments.

Other optional materials can be those materials approved for use in cosmetics and that are described in the CTFA Cosmetic Ingredient Handbook, Second Edition, The Cosmetic, Toiletries, and Fragrance Association, Inc. 1988, 1992.

The formulated personal care composition may be a shampoo, conditioner, body wash, shower gel, moisturiser, cosmetic, skin care formulations or beauty bar.

The form of the formulated personal care composition will depend on the intended use thereof.

It may be provided as a gel, paste, solid, liquid, emulsion or cream.

Preferably the formulated personal care composition comprises from 0.5 to 60 wt %, preferably from 1 to 40 wt %, suitably from 3 to 35 wt %, preferably from 5 to 30 wt %, for example from 6 to 20 wt % of one or more compounds of formula (II).

Preferably the formulated personal care composition comprises, in total, from 0.5 to 99 wt %, preferably from 1 to 80 wt % of active surfactant compounds. The amount of active surfactant compounds present in the composition will depend on the nature of the composition and the intended use thereof.

In some embodiments the formulated personal care composition may comprise, in total, from 5 to 50 wt %, or from 10 to 40 wt % of active surfactant compounds.

In some preferred embodiments the formulated personal care composition of the present invention is a transparent composition. A transparent composition preferably transmits at least 90% of light having a wavelength of 600 nm.

In some preferred embodiments the formulated personal care compositions of the present invention are substantially sulfate-free.

Preferably the formulated personal care compositions comprise less than 1 wt % sulfate based surfactants, preferably less than 0.5 wt %, suitably less than 0.1 wt %, preferably less than 0.05 wt %, more preferably less than 0.01 wt %, for example less than 0.001 wt % In some preferred embodiments the formulated personal care compositions of the present invention are low salt or salt-free.

Preferably the formulated personal care compositions comprise less than 1 wt % sodium chloride, preferably less than 0.5 wt %, suitably less than 0.1 wt %, preferably less than 0.05 wt %, more preferably less than 0.01 wt %, for example less than 0.001 wt %

According to an eighth aspect of the present invention there is provided a method of preparing a formulated composition of the sixth aspect, the method comprising preparing a surfactant composition according to the method of the first aspect and admixing said composition with one or more further components.

According to a ninth aspect of the present invention there is provided a method of preparing a formulated composition of the sixth aspect, the method comprising admixing a precursor concentrate composition of the seventh aspect with one or more further components.

The present invention offers significant advantage over the use of surfactants derived from petrochemical sources. As well as the obvious environmental benefits, the present invention may offer a reduction in the levels of impurities and reduced odours.

Surfactants provided by the present invention may also have an improved colour which is more desirable to users. For example the surfactant compositions may have a more consistent even colour and/or reduced yellowing.

The invention will now be described with reference to the following non-limiting examples.

### EXAMPLE 1

### % RCI Calculation

The percentage renewable carbon index (% RCI) was obtained by dividing the number of carbon atoms derived from renewable sources by the total number of carbon atoms. The % RCI of the compositions was obtained by taking a weighted average of the % RCI of each of the carbon-containing surfactant ingredients.

1. A method of preparing an alkyl acyl isethionate surfactant composition, the method comprising reacting a carboxylic acid of formula R¹COOH or a reactive equivalent thereof with a compound of formula (I): 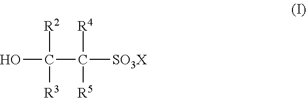
wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; and wherein the compound of formula (I) has a percentage renewable carbon index (RCI) of at least 50%.

2. The method according to claim 1 wherein the compound of formula (I) has a percentage renewable carbon index (RCI) of 95 to 100%.

3. The method according to claim 1, wherein the carboxylic acid of formula R¹COOH or a reactive equivalent thereof has a percentage renewable carbon index (RCI) of 95 to 100%.

4. A surfactant composition comprising one or more alkyl acyl isethionate compounds of formula (II): 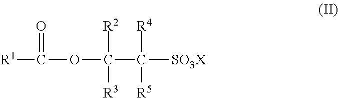
wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source.

5. A compound of formula (II): 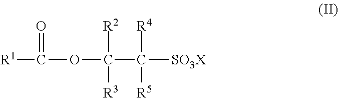
wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source.

6. A compound of formula (I): 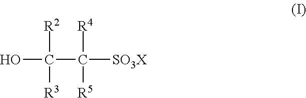
wherein X is hydrogen, a metal ion or an optionally substituted ammonium ion; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms in the compound are from a renewable source

7. The surfactant of claim 4, present in an effective amount for toilet care, automatic dishwashing, manual dishwashing, laundry, fabric care, kitchen care, carpet cleaning, air fresheners, vehicle care, polishing products, machine cleaning and maintenance, pesticides, insecticides, fungicides, herbicides, oilfield chemical applications, marine applications, personal care or institutional/industrial cleaning.

8. A formulated composition comprising:
a surfactant composition comprising one or more alkyl acyl isethionate compounds of formula (II): 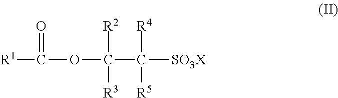
wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R⁵ is not hydrogen; wherein at least 50% of the carbon atoms provided by the moiety CR²R³CR⁴R⁵SO₃X in the compound of formula (II) are from a renewable source; and
one or more further components.

9. A precursor concentrate composition comprising:
a surfactant composition comprising one or more alkyl acyl isethionate compounds of formula (II): 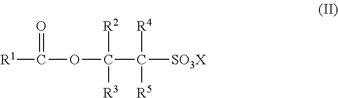
wherein X is hydrogen, a metal ion or an optionally substituted ammonium ion; R¹ represents an optionally substituted C₄-C₃₆ hydrocarbyl group; each of R², R³, R⁴ and R⁵ independently represents hydrogen or a C₁-C₄ alkyl group and wherein at least one of R², R³, R⁴ and R¹ is not hydrogen; wherein at least 50% of the carbon atoms provided by the moiety CR²R³CR⁴R⁵SO₃X in the compound of formula (II) are from a renewable source; and
one or more further surfactants.

10. The surfactant according to claim 4 wherein from 95 to 100% or 98 to 100% of the carbon atoms provided by the moiety CR²R³CR⁴R⁵SO₃X in the compound of formula (II) are from a renewable source.

11. The surfactant according to claim 4, wherein the compound of formula (II) has a percentage renewable carbon index (% RCI) of from 95 to 100%, preferably from 99 to 100%.

12. The method according to claim 1, wherein R¹ is provided by one or more naturally occurring fatty acids.

13. The method to any preceding claim 1, wherein R¹ is provided by one or more fatty acids selected from coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil.

14. The method according to claim 1, wherein one of the groups R², R³, R⁴ and R⁵ represents an optionally substituted C₁-C₄ alkyl group and the remaining groups represent hydrogen.

15. The method according to claim 1, further comprising a mixture of isomers comprising a compound of formula R¹COOCH₂CHR⁴SO₃X in which R⁴ represents a C₁-C₄ alkyl group (preferably methyl) and a compound of formula R¹COOCHR₂CH₂SO₃X in which R² represents a C₁-C₄ alkyl group (preferably methyl); preferably wherein in approximately 90% of compounds R² is methyl and R⁴ is hydrogen and in approximately 10% of compounds R² is hydrogen and R⁴ is methyl.

16. The formulated composition according to claim 8, wherein the one or more further components are selected from chelating agents, preservatives, pH modifiers, hydrotropes and further surfactants.

17. The formulated composition according to claim 16, further comprising one or more further surfactants.

18. The formulated composition according to claim 17, wherein the total percentage RCI of the formulated composition is at least 80%.

19. The method of preparing a formulated composition according to claim 8, the method comprising preparing a surfactant composition according to the method of claim 1 and admixing said composition with one or more further components.

20. The precursor concentrate composition according to claim 9, wherein the total percentage RCI of precursor concentrate composition is at least 80%.

21. The method of preparing a formulated composition according to claim 8, the method comprising admixing a precursor concentrate composition of claim 9 with one or more further components.
